(19) United States
(12) Patent Application Publication
(10) Pub. No.: US 2007/0031893 A1
(43) Pub. Date: Feb. 8, 2007

(54) Method for measuring reaction rate coefficient in analysis utilizing total reflection attenuation

(75) Inventor: Masato Some, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/496,483

(22) Filed: Aug. 1, 2006

(30) Foreign Application Priority Data
Aug. 1, 2005 (JP) 222953/2005

(51) Int. Cl. G06F 19/00; G01N 33/53

(52) U.S. Cl. 435/7.1; 702/19; 702/22

(57) ABSTRACT

An object of the present invention is to provide a method for measuring a reaction rate coefficient in an analysis utilizing total reflection attenuation, which is capable of calculating the reaction rate coefficient speedily and accurately. The present invention provides a method for measuring adsorption rate coefficient (Ka) and diffusion coefficient (D) in a reaction between an analyte molecule immobilized on a metal surface and a molecule that interacts with the analyte molecule, by measuring an angular change in the total reflection attenuation angle (θSP) using an analysis device utilizing total reflection attenuation, which comprises (1) providing multiple simulation curves of a binding dissociation reaction for sets of variables in which adsorption rate coefficient (Ka) and diffusion coefficient (D) are each varied within a predetermined width, (2) preparing a measurement curve of the binding dissociation reaction based on an angular change in a measured total reflection attenuation angle (θSP), (3) examining the level of correspondence between the measurement curve prepared in above (2) and the multiple simulation curves of above (1), and (4) applying the adsorption rate coefficient (Ka) and diffusion coefficient (D) that were used for the preparation of the simulation curve with the highest level of correspondence to the adsorption rate coefficient (Ka) and the diffusion coefficient (D) in the reaction between the analyte molecule immobilized on the metal surface and the molecule that interacts with the analyte molecule.

### TECHNICAL FIELD

The present invention relates to a method for measuring the rate coefficient of the reaction between an analyte molecule immobilized on a metal surface and a molecule that interacts with the analyte molecule in analysis utilizing total reflection attenuation (for example, surface plasmon resonance analysis, leakage mode analysis and the like).

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The QCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules. An example of a surface plasmon resonance measurement device is the device described in Japanese Patent Laid-Open (Kokai) No. 2001-330560.

When a specific binding reaction between a physiologically active substance and a test substance is measured, the binding reaction is generally measured by: connecting in series a reference cell, to which a physiologically active substance interacting with a test substance does not bind, with a detection cell, to which a physiologically active substance interacting with a test substance binds; placing the connected cells in a flow channel system; and feeding a liquid through the reference cell and the detection cell, so as to carry out the measurement of the binding reaction. During the measurement, the liquid contained in the above flow channel system is exchanged from a reference liquid containing no test substance to be measured to a sample liquid containing a test substance to be measured, so as to cause the binding reaction between the physiologically active substance and the test substance to be initiated, and to measure a change in signals due to a lapse of time.

As mentioned above, a biosensor that uses surface plasmon resonance detects the binding of an analyte to a sensor (a metal film and a ligand) as a change in the refractive index (and an angular change of a dark line caused thereupon). If the time is plotted on the horizontal axis and the binding signal is plotted on the vertical axis, a signal (indicating the amount of binding or the like) that is referred to as a so-called “sensorgram” can be observed with the elapse of time. It is important to carry out fitting of the following rate equation (i) to the sensorgram followed by determination of the rate coefficients such as the adsorption rate coefficient (Ka) and the dissociation rate coefficient (Kd). Such procedures are extensively adopted in the field of drug screening.
dR/dt=Kₐ×C×{Rmax−R(t)}−K_{d}×R(t) (i)
R(t)=(Kₐ×C×Rmax)/(Kₐ×C+K_{d})×(1−exp(−Kₐ×C+K_{d})×t)) (ii)
(the result of solving equation (i))
wherein Kₐ represents an adsorption rate coefficient; K_{d} represents a dissociation rate coefficient; C represents an analyte concentration (known); Rₘₐₓ represents the theoretical maximum amount of binding; and t represents a time.

As described above, the binding dissociation reaction measured with an SPR signal is expressed by differential equations of time and space that employ variables such as adsorption rate coefficient (Ka), dissociation rate coefficient (Kd), diffusion coefficient (D), and theoretical maximum binding amount (Rmax). Thus, it is possible to create a binding dissociation reaction curve by dividing time and space at fine intervals when the values of Ka, Kd, D, Rmax are determined, and then performing a simulation. However, it is difficult to inversely calculate Ka, Kd, D, and Rmax from the binding dissociation reaction curve, and any potential approximation formula for the back calculation would have accuracy problems.

A possible solution is to create a reaction curve by simulation while Ka, Kd, D, Rmax are varied with respect to a measured binding dissociation curve, and to adopt the values of a simulated reaction curve that matched the measured curve. However, in this system, it could take as long as several to dozens of hours to obtain a single reaction curve by simulation. Thus, the method whereby a reaction curve is created by simulation by varying the variables on an ad-hoc basis is not practical.

### DISCLOSURE OF THE INVENTION

It is an object of the present invention to provide a method for measuring a reaction rate coefficient in an analysis utilizing total reflection attenuation, which is capable of calculating the reaction rate coefficient speedily and accurately. The present inventors conducted concentrated studies in order to attain the above object. As a result, they have found that the rate coefficients can be speedily and accurately calculated by obtaining a reaction curve simulation data in an analysis utilizing total reflection attenuation, storing it in tables, and then examining the level of correspondence between the simulation data and an actually measured reaction curve. This has led to the completion of the present invention.

Namely, the present invention provides a method for measuring adsorption rate coefficient (Ka) and diffusion coefficient (D) in a reaction between an analyte molecule immobilized on a metal surface and a molecule that interacts with the analyte molecule, by measuring an angular change in the total reflection attenuation angle (θSP) using an analysis device utilizing total reflection attenuation, which comprises
(1) providing multiple simulation curves of a binding dissociation reaction for sets of variables in which adsorption rate coefficient (Ka) and diffusion coefficient (D) are each varied within a predetermined width,
(2) preparing a measurement curve of the binding dissociation reaction based on an angular change in a measured total reflection attenuation angle (θSP),
(3) examining the level of correspondence between the measurement curve prepared in above (2) and the multiple simulation curves of above (1), and
(4) applying the adsorption rate coefficient (Ka) and diffusion coefficient (D) that were used for the preparation of the simulation curve with the highest level of correspondence to the adsorption rate coefficient (Ka) and the diffusion coefficient (D) in the reaction between the analyte molecule immobilized on the metal surface and the molecule that interacts with the analyte molecule.

Preferably, in the step (1), multiple simulation curves of a binding dissociation reaction may be provided for sets of variables in which adsorption rate coefficient (Ka), dissociation rate coefficient (Kd), diffusion coefficient (D), theoretical maximum binding amount (Rmax), and C (analyte concentration) are each varied within a predetermined width.

Preferably, the level of correspondence between the measurement curve and the simulation curve may be examined by using a square sum of the error as an index.

Preferably, using an analysis device utilizing total reflection attenuation and comprising a flow channel system having a cell formed on a metal film and a light-detecting means for detecting a total reflection attenuation angle (θSP) by measuring the intensity of a light beam totally reflected on the metal film, an angular change in the total reflection attenuation angle (θSP) can be measured in a state where the liquid flow is stopped, after liquids in the flow channel are exchanged.

Preferably, there is used a analysis device utilizing total reflection attenuation, which comprises a dielectric block, a metal film formed on one side of the dielectric block, a light source for generating a light beam, an optical system for allowing the above light beam to enter the above dielectric block so that total reflection conditions can be obtained at the interface between the dielectric block and the metal film and so that various incidence angles can be included, a flow channel system comprising a cell formed on the above metal film, and a light-detecting means for detecting a total reflection attenuation angle (θSP) by measuring the intensity of a light beam totally reflected at the above interface.

Preferably, the liquid contained in the above flow channel system is exchanged from a reference liquid containing no test substance to be measured to a sample liquid containing a test substance to be measured, and thereafter an angular change in the total reflection attenuation angle (θSP) can be measured in a state where the sample liquid flow is stopped.

Preferably, as the range in which the level of correspondence between the measurement curve and the simulation curve is examined, a part of a binding signal curve, a part of a dissociation signal curve, or both thereof can be used.

Preferably, a signal change in surface plasmon resonance can be measured by using a surface plasmon resonance measurement device.

### BRIEF DESCRIPTION OF THE DRAWINGS

FIG. 1 shows a surface plasmon resonance measurement device used in the examples.

FIG. 2 shows a dielectric block used in the examples.

FIG. 3 shows measurement data obtained in the Example and 16 items of simulation data.

FIG. 4 shows an enlarged view of FIG. 3 (showing the portions where 170<x<200 and 350<y<400).

In figures, 10 indicates measurement unit, 11 indicates dielectric block, 12 indicates metal film, 13 indicates sample-retaining frame, 14 indicates sensing substance, 30 indicates light beam, 31 indicates laser light source, 32 indicates condenser lens, 40 indicates light detector, S40 indicates output signal, 400 indicates guide rod, 401 indicates slide block, 402 indicates precision screw, 403 indicates pulse motor, 404 indicates motor controller, 410 indicates unit connector, and 411 indicates connecting member.

### BEST MODE FOR CARRYING OUT THE INVENTION

Hereafter, embodiments of the present invention are described.

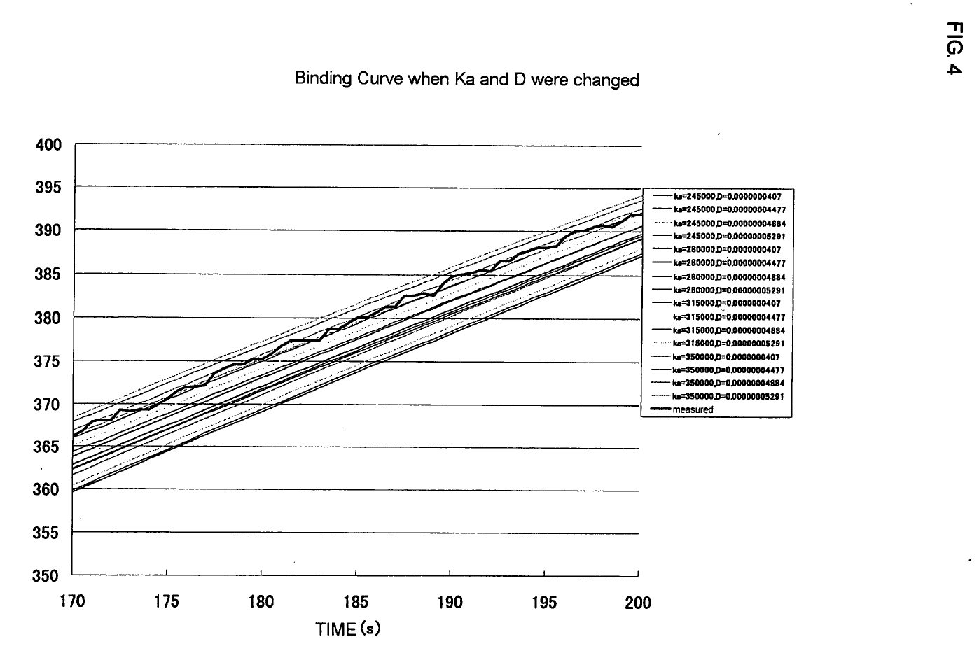

In accordance with the method of the present invention, simulation curves of a binding dissociation reaction are prepared in advance for each set of variables in which Ka, Kd, D, or Rmax is varied at proper intervals, and the simulation curves are stored in tables (see FIG. 3, for example). Then, the level of correspondence between an actually measured binding dissociation reaction curve and the stored table data is examined. Then, a group of variables used for the preparation of the reaction curve of which the level of correspondence is highest can be considered to be the solution to the measured data.

In reality, Kd can be determined from the dissociating portion of the binding dissociation, and Rmax can be calculated on an ad-hoc basis because its only effect is to produce an integer multiple of the whole. Thus, it is sufficient to provide tables in which the two parameters Ka and D are varied.

For obtaining data with the highest level of correspondence from the tables, there are three possible methods, as follows:

(1) Round-robin method (all of the data in the tables is tried).

(2) Two-stage search method (tables are roughly searched, and then portions where the highest data seems likely to be found are further investigated).

(3) Non-linear optimization (variables are varied such that the level of correspondence becomes higher).

Any of these methods can be selected depending on the presence of a local solution and the calculation time. The round-robin method, the two-stage search method, and the non-linear search method are described below.

(Round-robin Method)

For example, the five parameters, namely, adsorption rate coefficient (Ka), dissociation rate coefficient (Kd), diffusion coefficient (D), C (analyte concentration), and theoretical maximum binding amount (Rmax) are labeled as a, α, β, γ, δ, and θ, respectively, for simplicity's sake. For example, when each parameter consists of 100 parameter items, the number of parameters would be 100×100×100×100×100=10¹⁰. In the round-robin method, these parameters are searched for optimum parameters sequentially in lexicographical order, such as:
(α1, β1, γ1, δ1, θ1)→(α2, β1, γ1, δ1, θ1)→(α3, β1, γ1, δ1, θ1)→, . . .
(α1, β2, γ1, δ1, θ1)→(α2, β2, γ1, δ1, θ1)→(α3, β2, γ1, δ1, θ1)→, . . . , and so on.
(Two-stage Search Method)

For example, αi, βj, γn, δ1, θm are initially examined coarsely by limiting the i, j, n, 1 and m to the multiples of 10. In this case, the first stage of analysis ends in a small number of times, namely, 10×10×10×10×10=10⁵. As a result, if it is found that (αp, βq, γr, δs, θt) provide good results, then optimum parameters are searched for in α(p−5) to α(p+4), β(q−5) to β(q+4), . . . , or the like, namely 10×10×10×10×10=10⁵ sets. Thus, the optimum parameters can be retrieved within 2×10⁵ times, so that the required amount of time can be reduced.

(Non-linear Optimization)

For example, if we start with (αi, βi, γi, δi, θi), a is examined for (α(i−1), αi, and α(i+1)), β, is examined for (β(i−1), βi, and β(i+1)), . . . , and so on, thus examining 3×3×3×3×3=3⁵ items of data. Then, if it is found that, among those data items, (αi, β(i+1), γ(i+1), δi, θi) provide good results, then the data can be examined within ranges such as (α(i−1), αi, α(i+1)) for α, (βi, β(i+1), β(i+2)) for β, (γi, γ(i+1), γ(i+2)) for γ, β(i−1), δi, δ(i+1)) for δ, and so on. Thus, in the non-linear optimization (direct search method), the data is searched by comparing data items that precede and follow central points until no better results can be obtained. Other examples of the non-linear optimization method, in addition to the above-described direct search method, include the gradient method and the inverse matrix calculation method (“Waveform Data Processing for Scientific Measurement”, edited by Shigeo Minami, CQ Publishing Company, pp. 182-183).

Possible method for calculating the level of correspondence between a measurement curve and a simulation curve is to find the square sum of the error. The smaller the value of the square sum, the higher the level of correspondence.

Furthermore, if there are some aspects that need to be given particular importance upon examining the level of correspondence, weighting may be provided instead of just adding up the square of the error.

The present invention relates to a method for calculating the rate coefficient between an analyte molecule immobilized on a metal surface and a molecule that interacts with the analyte molecule. For example, changes in the surface plasmon resonance signal can be detected using a surface plasmon resonance measurement device comprising a flow channel system having a cell formed on a metal film and a light-detecting means for detecting the state of surface plasmon resonance by measuring the intensity of a light beam totally reflected on the metal film, in a state where the flow of the liquid has been stopped, after the liquid contained in the above flow channel system has been exchanged.

As time passes, an analyte molecule is adsorbed on a molecule that interacts with the analyte molecule which was immobilized on a metal surface. This phenomenon can be described by the following equation (1):
dθ/dt=kₐ×cₛ×(1−θ)−k_{d}×θ (1)
wherein θ is the rate of adsorption (=adsorbed amount/saturation adsorbed amount), kₐ is an adsorption rate coefficient, k_{d} is a dissociation rate coefficient, and cₛ is the concentration of the analyte molecule in the vicinity of the metal surface.

In an ideal condition where the metal surface can be constantly supplied with fresh solution, cₛ becomes constant, and kₐ and k_{d} can be determined from the measurement results by solving simple differential equations.

However, the flow on the metal surface is very slow, and, if cₛ is to be kept constant, the analyte molecule solution needs to flow at high rate. On the other hand, regarding the surface plasmon, disturbance in the flow on the metal surface causes fluctuations in a signal, and a large volume of the analyte molecule needs to be used if the rate of flow is to be increased. For these reasons, it is impossible to keep cₛ constant in practice.

When cₛ is not constant, the change in concentration due to the adsorption/dissociation of the analyte molecule becomes a function that is varied by the diffusion of the analyte molecule from offshore. The diffusion in this case can be expressed by the following equation (2):
∂c/∂t=D×∂²c/∂x² (2)
wherein x is the distance from the metal surface, D is the diffusion coefficient of the analyte molecule, and c is the concentration of the analyte molecule, where c=cₛ when x=0).

Meanwhile, it is known that a surface plasmon signal R (the difference from a surface plasmon signal when the analyte molecule is not adsorbed) is proportional to the surface-adsorbed amount of the analyte molecule, and it is expressed by the following equation (3):
θ=R/Rmax (3)
wherein θ is the rate of adsorption (=adsorbed amount/saturation adsorbed amount), R is a surface plasmon signal, and Rmax is a signal when the analyte molecule is saturation-adsorbed).

By using the above equations (1), (2), and (3), it becomes possible to create simulation curves of each binding dissociation reaction for sets of variables in which Ka, Kd, D, or Rmax are varied at proper intervals.

In the present invention, a change in surface plasmon resonance may be measured in a state where the flow of a liquid has been stopped, so that the noise width of a change in signals of a reference cell during measurement and base line fluctuation can be suppressed, and thus binding detection data with high reliability can be obtained. The time of the stop of the flow of the liquid is not particularly limited. For example, it may be between 1 second and 30 minutes, preferably between 10 seconds and 20 minutes, and more preferably between 1 minute and 20 minutes.

In the present invention, preferably, the liquid contained in a flow channel system is exchanged from a reference liquid containing no test substance to be measured to a sample liquid containing a test substance to be measured, and thereafter, a change in surface plasmon resonance can be measured in a state where the flow of the sample liquid has been stopped.

In the present invention, preferably, a reference cell, to which a substance interacting with a test substance does not bind, is connected in series with a detection cell, to which a substance interacting with a test substance binds, the connected cells are placed in a flow channel system, and a liquid is then fed through the reference cell and the detection cell, so that a change in surface plasmon resonance can be measured.

In addition, in the present invention, the ratio (Ve/Vs) of the amount of a liquid exchanged (Ve ml) in a single measurement to the volume (Vs ml) of a cell used in measurement (and when the aforementioned reference cell and detection cell are used, the total volume of these cells) is preferably between 1 and 100. Ve/Vs is more preferably between 1 and 50, and particularly preferably between 1 and 20. The volume (Vs ml) of a cell used in measurement is not particularly limited. It is preferably between 1×10⁻⁶ and 1.0 ml, and particularly preferably between 1×10⁻⁵ and 1×10⁻¹ ml. The period of time necessary for exchanging the liquid is preferably between 0.01 second and 100 seconds, and particularly preferably between 0.1 second and 10 seconds.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light. The surface plasmon resonance measurement device used in the present invention will be described below.

The surface plasmon resonance measurement device is a device for analyzing the properties of a substance to be measured using a phenomenon whereby a surface plasmon is excited with a lightwave. The surface plasmon resonance measurement device used in the present invention comprises a dielectric block, a metal film formed on a face of the dielectric block, a light source for generating a light beam, an optical system for allowing the above light beam to enter the above dielectric block such that total reflection conditions can be obtained at the interface between the above dielectric block and the above metal film and that components at various incident angles can be contained, and a light-detecting means for detecting the state of surface plasmon resonance by measuring the intensity of the light beam totally reflected at the above interface.

Moreover, as stated above, the above dielectric block is formed as one block comprising the entity of the entrance face and exit face of the above light beam and a face on which the above metal film is formed, and the above metal film is integrated with this dielectric block.

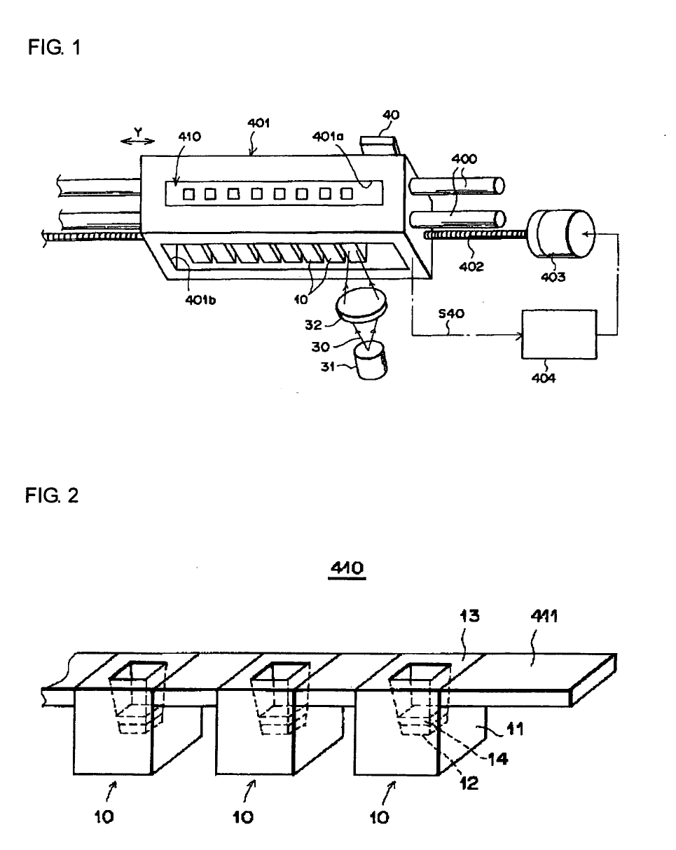

In the present invention, more specifically, a surface plasmon resonance measurement device shown in FIGS. 1 to 32 of Japanese Patent Laid-Open No. 2001-330560, and a surface plasmon resonance device shown in FIGS. 1 to 15 of Japanese Patent Laid-Open No. 2002-296177, can be preferably used. All of the contents as disclosed in Japanese Patent Laid-Open Nos. 2001-330560 and 2002-296177 cited in the present specification are incorporated herein by reference as a part of the disclosure of this specification.

For example, the surface plasmon resonance measurement device described in Japanese Patent Laid-Open No. 2001-330560 is characterized in that it comprises: a dielectric block; a thin metal film formed on a face of the dielectric block; multiple measurement units comprising a sample-retaining mechanism for retaining a sample on the surface of the thin film; a supporting medium for supporting the multiple measurement units; a light source for generating a light beam; an optical system for allowing the above light beam to enter the dielectric block at various angles so that total reflection conditions can be obtained at the interface between the dielectric block and the metal film; a light-detecting means for measuring the intensity of the light beam totally reflected at the above interface and detecting the state of total reflection attenuation caused by surface plasmon resonance; and a driving means for making the above supporting medium, the above optical system and the above light-detecting means move relative to one another, and successively placing each of the above multiple measurement units in a certain position appropriate to the above optical system and the above light-detecting means, so that the above total reflection conditions and various incident angles can be obtained with respect to each dielectric block of the above multiple measurement units.

It is to be noted that in the above measurement device, the above optical system and light-detecting means are kept in a resting state and the above driving means makes the above supporting medium move.

In such a case, the above supporting medium is desirably a turntable for supporting the above multiple measurement units on a circle centered on a rotation axis, and the above driving means is desirably a means for intermittently rotating this turntable. In this case, a medium for supporting the above multiple measurement units that are linearly arranged in a line may be used as the above supporting medium, and a means that makes such a supporting medium move linearly in an intermittent fashion in the direction in which the above multiple measurement units are arranged may be applied as the above driving means.

Otherwise, on the contrary, it may also be possible that the above supporting medium be retained in a resting state and that the above driving means makes the above optical system and light-detecting means move.

In such a case, the above supporting medium is desirably a medium for supporting the above multiple measurement units on a circle, and the above driving means is desirably a means for intermittently rotating the above optical system and light-detecting means along the multiple measurement units supported by the above supporting medium. In this case, a medium for supporting the above multiple measurement units that are linearly arranged in a line may be used as the above supporting medium, and a means that makes the above optical system and light-detecting means move linearly in an intermittent fashion along the multiple measurement units supported by the above supporting medium may be applied as the above driving means.

Otherwise, when the above driving means has a rolling bearing that supports a rotation axis, the driving means is desirably configured such that after the rotation axis has been rotated to a certain direction and a series of measurements for the above multiple measurement units has been terminated, the above rotation axis is equivalently rotated to the opposite direction, and then it is rotated again to the same above direction for the next series of measurements.

In addition, the above-described measurement device is desirably configured such that the above multiple measurement units are connected in a line with a connecting member so as to constitute a unit connected body and that the above supporting medium supports the unit connected body.

Moreover, in the above-described measurement device, it is desirable to establish a means for automatically feeding a given sample to each sample-retaining mechanism of the multiple measurement units supported by the above supporting medium.

Furthermore, in the above-described measurement device, it is desirable that the dielectric block of the above measurement unit be immobilized to the above supporting medium, that a thin film layer and a sample-retaining mechanism of the measurement unit be unified so as to constitute a measurement chip, and that the measurement chip be formed such that it is exchangeable with respect to the above dielectric block.

When such a measurement chip is applied, it is desirable to establish a cassette for accommodating a multiple number of the measurement chips and a chip-supplying means for successively taking a measurement chip out of the cassette and supplying it in a state in which it is connected to the above dielectric block.

Otherwise, it may also be possible to unify the dielectric block of the measurement unit, the thin film layer and the sample-retaining mechanism, so as to constitute a measurement chip, and it may also be possible for this measurement chip to be formed such that it is exchangeable with respect to the above supporting medium.

When a measurement chip has such a structure, it is desirable to establish a cassette for accommodating a multiple number of measurement chips and a chip-supplying means for successively taking a measurement chip out of the cassette and supplying it in a state in which it is supported by the supporting medium.

The above optical system is desirably configured such that it makes a light beam enter the dielectric block in a state of convergent light or divergent light. Moreover, the above light-detecting means is desirably configured such that it detects the position of a dark line generated due to attenuated total reflection, which exists in the totally reflected light beam.

Furthermore, the above optical system is desirably configured such that it makes a light beam enter the above interface in a defocused state. In this case, the beam diameter of the light beam at the above interface in a direction wherein the above supporting medium moves is desirably ten times or greater the mechanical positioning precision of the above supporting medium.

Still further, the above-described measurement device is desirably configured such that the measurement unit is supported on the upper side of the above supporting medium, such that the above light source is placed so as to project the above light beam from a position above the above supporting medium to downwards, and such that the above optical system comprises a reflecting member for reflecting upwards the above light beam projected to downwards as described above and making it proceed towards the above interface.

Still further, the above-described measurement device is desirably configured such that the above measurement unit is supported on the upper side of the above supporting medium, such that the above optical system is constituted so as to make the above light beam enter the above interface from the downside thereof, and such that the above light-detecting means is placed in a position above the above supporting medium with a light-detecting plane thereof facing downwards, as well as comprising a reflecting member for reflecting upwards the totally reflected light beam at the above interface and making it proceed towards the above light-detecting means.

What is more, the above-described measurement device desirably comprises a temperature-controlling means for maintaining the temperature of the above measurement unit before and/or after being supported by the above supporting medium at a predetermined temperature.

Moreover, the above-described measurement device desirably comprises a means for stirring the sample stored in the sample-retaining mechanism of the measurement unit supported by the above supporting medium before detecting the state of attenuated total reflection as mentioned above.

Furthermore, in the above-described measurement device, it is desirable to establish in at least one of the multiple measurement units supported by the above supporting medium a standard solution-supplying means for supplying a standard solution having optical properties associated with the optical properties of the above sample, as well as a correcting means for correcting data regarding the above attenuated total reflection state of the sample based on the data regarding the above attenuated total reflection state of the above standard solution.

In such a case, if the sample is obtained by dissolving a test substance in a solvent, it is desirable that the above standard solution-supplying means be a means for supplying the above solvent as a standard solution.

Still further, the above measurement device desirably comprises: a mark for indicating individual recognition information; a reading means for reading the above mark from the measurement unit used in measurement; an inputting means for inputting sample information regarding the sample supplied to the measurement unit; a displaying means for displaying measurement results; and a controlling means connected to the above displaying means, inputting means and reading means, which stores the above individual recognition information and sample information of each measurement unit while associating them with each other, as well as making the above displaying means display the measurement results of the sample retained in a certain measurement unit while associating them with the above individual recognition information and sample information of each measurement unit.

When a substance interacting with a physiologically active substance is detected or measured using the above-described measurement device, a state of attenuated total reflection is detected in a sample contained in one of the above measurement units, and thereafter, the above supporting medium, optical system and light-detecting means are moved relative to one another, so that a state of attenuated total reflection is detected in a sample contained in another measurement unit. Thereafter, the above supporting medium, optical system and light-detecting means are again moved relative to one another, so that a state of attenuated total reflection is detected again the sample contained in the above one measurement unit, thereby completing the measurement.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which a total reflection attenuation angle (θSP) has changed is measured.

Moreover, Japanese Patent Laid-Open No. 2001-330560 describes a measurement device using total reflection attenuation, which involves successively measuring multiple measurement chips mounted on a turntable or the like, so as to measure many samples in a short time.

The measurement chip used in the present invention is used for the surface plasmon resonance measurement device or the leaking mode measurement device which have a structure described herein (these are referred to as measurement device utilizing total reflection attenuation), and comprises a dielectric block and a metal film formed on a face of the dielectric block, in which the dielectric block is formed as one block comprising the entirety of the entrance face and exit face of the light beam and a face on which the above metal film is formed, the above metal film is integrated with the above dielectric block.

A metal constituting the metal film is not particularly limited, as long as surface plasmon resonance is generated. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 0.1 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and having excellent workability are preferably used.

Preferably, the metal film has a functional group capable of immobilizing a physiologically active substance on the outermost surface of the substrate. The term “the outermost surface of the substrate” is used to mean “the surface, which is farthest from the substrate”.

Examples of a preferred functional group may include —OH, —SH, —COOH, —NR¹R² (wherein each of R¹ and R² independently represents a hydrogen atom or lower alkyl group), —CHO, —NR³NR¹R² (wherein each of R¹, R² and R³ independently represents a hydrogen atom or lower alkyl group), —NCO, —NCS, an epoxy group, and a vinyl group. The number of carbon atoms contained in the lower alkyl group is not particularly limited herein. However, it is generally about C1 to C10, and preferably C1 to C6.

Examples of the method of introducing such a functional group include a method which involves applying a polymer containing a precursor of such a functional group on a metal surface or metal film, and then generating the functional group from the precursor located on the outermost surface by chemical treatment.

In the measurement chip obtained as mentioned above, a physiologically active substance is covalently bound thereto via the above functional group, so that the physiologically active substance can be immobilized on the metal film.

A physiologically active substance immobilized on the surface for the measurment chip of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

When a physiologically active substance is a protein such as an antibody or enzyme, or nucleic acid, an amino group, thiol group or the like of the physiologically active substance is covalently bound to a functional group located on a metal surface, so that the physiologically active substance can be immobilized on the metal surface.

A measurement chip to which a physiologically active substance is immobilized as described above can be used to detect and/or measure a substance which interacts with the physiologically active substance.

For example, at least a measurement chip (cell), to the surface of which a physiologically active substance binds by covalent bonding is used; a sample liquid containing a test substance to be measured is allowed to come into contact with the above cell; and after exchanging the liquid contained in a flow channel system, a change in surface plasmon resonance can be measured in a state where the flow of the liquid has been stopped. As a test substance, a sample containing a substance interacting with the aforementioned physiologically active substance can be used, for example.

The present invention is further described in detail by the following examples, but the present invention is not limited by the examples.

### EXAMPLES

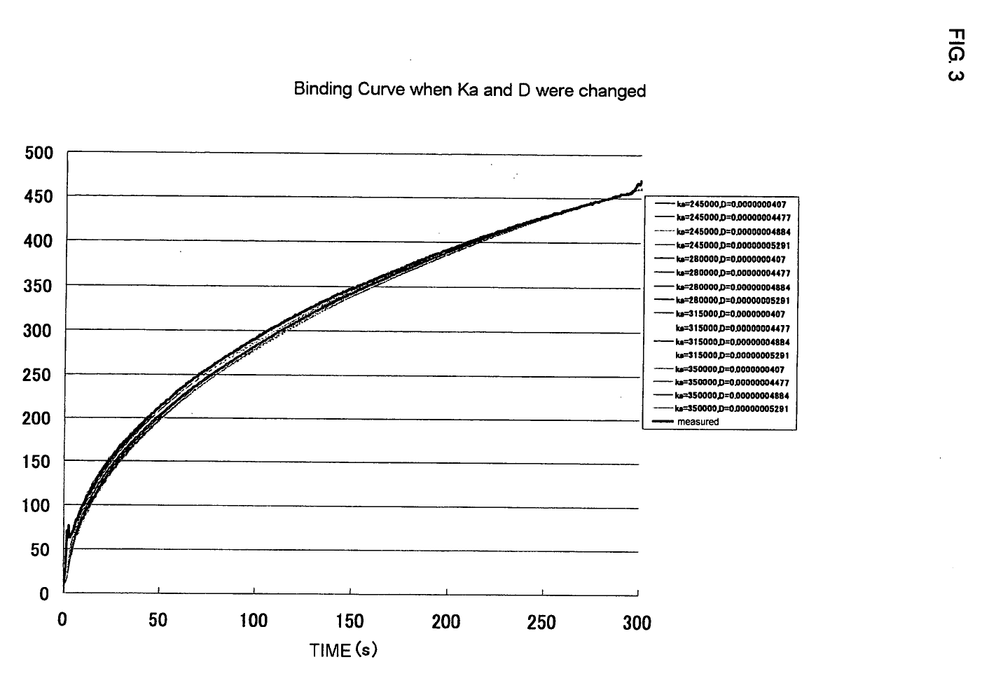

The following experiment was carried out using a device shown in FIG. 22 of Japanese Patent Laid-Open No. 2001-330560 (hereinafter referred to as the surface plasmon resonance measurement device of the present invention) (shown in FIG. 1 of the present specification) and a dielectric block shown in FIG. 23 of Japanese Patent Laid-Open No. 2001-330560 (hereinafter referred to as the dielectric block of the present invention) (shown in FIG. 2 of the present specification).

In the surface plasmon resonance measurement device shown in FIG. 1, a slide block 401 is used as a supporting medium for supporting measurement units, which is joined to two guide rods 400, 400 placed in parallel with each other while flexibly sliding in contact, and which also flexibly moves linearly along the two rods in the direction of an arrow Y in the figure. The slide block 401 is screwed together with a precision screw 402 placed in parallel with the above guide rods 400, 400, and the precision screw 402 is reciprocally rotated by a pulse motor 403 which constitutes a supporting medium-driving means together with the precision screw 402.

It is to be noted that the movement of the pulse motor 403 is controlled by a motor controller 404. This is to say, an output signal S 40 of a linear encoder (not shown in the figure), which is incorporated into the slide block 401 and detects the position of the slide block 401 in the longitudinal direction of the guide rods 400, 400, is inputted into the motor controller 404. The motor controller 404 controls the movement of the pulse motor 403 based on the signal S 40.

Moreover, below the guide rods 400, 400, there are established a laser light source 31 and a condenser 32 such that they sandwich from both sides the slide block 401 moving along the guide rods, and a photodetector 40. The condenser 32 condenses a light beam 30. In addition, the photodetector 40 is placed thereon.

In this embodiment, a stick-form unit connected body 410 obtained by connecting and fixing eight measurement units 10 is used as an example, and the measurement units 10 are mounted on the slide block 401 in a state in which eight units are arranged in a line.

FIG. 2 shows the structure of the unit connected body 410 in detail. As shown in the figure, the unit connected body 410 is obtained by connecting the eight measurement units 10 by a connecting member 411.

This measurement unit 10 is obtained by molding a dielectric block 11 and a sample-retaining frame 13 into one piece, for example, using transparent resin or the like. The measurement unit constitutes a measurement chip that is exchangeable with respect to a turntable. In order to make the measurement chip exchangeable, for example, the measurement unit 10 may be fitted into a through-hole that is formed in the turntable. In the present example, a sensing substance 14 is immobilized on a metal film 12.

(1) Preparation of Dextran Measurement Chip:

The dielectric block of the present invention, onto which gold having a thickness of 50 nm had been evaporated as a metal film, was treated with a Model-208 UV-ozone cleaning system (TECHNOVISION INC.) for 30 minutes. Thereafter, 5.0 mM solution containing 11-hydroxy-1-undecanethiol in ethanol/water (80/20) was added to the metal film such that it came into contact with the metal film, followed by a surface treatment at 25° C. for 18 hours. Thereafter, the resultant product was washed with ethanol 5 times, with a mixed solvent consisting of ethanol and water 1 time, and then with water 5 times.

Subsequently, the surface coated with 11-hydroxy-1-undecanethiol was allowed to come into contact with 10% by weight of epichlorohydrin solution (solvent: a mixed solution consisting of 0.4 M sodium hydroxide and diethylene glycol dimethyl ether at a ratio of 1 : 1), and a reaction was then carried out in a shaking incubator at 25° C. for 4 hours. Thereafter, the surface was washed with ethanol 2 times, and then with water 5 times.

Subsequently, 4.5 ml of 1 M sodium hydroxide was added to 40.5 ml of 25% by weight of dextran (T500, Pharmacia) aqueous solution. The obtained solution was allowed to come into contact with the surface treated with epichlorohydrin. The surface was then incubated in a shaking incubator at 25° C. for 20 hours. Thereafter, the resultant surface was washed with 50° C. water 10 times.

Subsequently, a mixture obtained by dissolving 3.5 g of bromoacetic acid in 27 g of a 2 M sodium hydroxide solution was allowed to come into contact with the above dextran-treated surface, and the obtained surface was then incubated in a shaking incubator at 28° C. for 16 hours. The surface was washed with water. Thereafter, the above-describe procedure was repeated once.

(2) Preparation of a Chip Having Protein A Immobilized thereon:

After a solution in the aforementioned dextran measurement chip was removed, 70 μl of a mixed solution of 200 mM EDC (N-ethyl-N′-(3-dimethylaminopropyl)carbodiimide hydrochloride) and 50 mM NHS (N-hydroxysuccinimide) was added thereto, and the resultant was then allowed to stand for 10 minutes. After the mixed solution was removed, the chip was washed three times with 100 μl of water and then three times with 100 μl of acetate 5.0 buffer (BIAcore). The chip was mounted on the surface plasmon resonance measurement device of the present invention while containing 100 μl of acetate 5.0 buffer, the inside of the chip was replaced with a protein A solution (a solution of 50 μ/ml protein A (Nacalai Tesque Inc.) in acetate 5.0 buffer (BIAcore)), the chip was allowed to stand for 30 minutes, and protein A was immobilized thereon. The inside of the chip was replaced with 1 M ethanolamine solution and the chip was allowed to stand for 3 minutes. The inside of the chip was washed ten times with 100 μl of acetate 5.0 buffer. Change in the resonance signal resulting from protein A immobilization was 500 RU.

(3) Preparation of a Control Chip:

After a solution in the dextran measurement chip prepared in (1) above was removed, 70 μl of a mixed solution of 200 mM EDC and 50 mM NHS was added thereto, and the resultant was then allowed to stand for 10 minutes. After the mixed solution was removed, the chip was washed three times with 100 μl of water and then three times with 100 μl of acetate 5.0 buffer. The inside of the chip was replaced with 1M ethanolamine solution and the chip was allowed to stand for 10 minutes. The inside of the chip was washed ten times with 100 μl of acetate 5.0 buffer.

(4) Preparation of a Flow Channel System:

A dielectric block was mounted on the chip having protein A immobilized thereon according to the present invention, and sealed with silicon rubber to prepare a cell having an inner volume of 15 μl. Two holes of a diameter of 1 mm were provided on the silicon rubber seal, and these two holes were connected with each other via a tefron tube having an inner diameter of 0.5 mm and an outer diameter of 1 mm to prepare a flow channel. Similarly, a cover and a flow chaneel were provided for the control chip, these two chips were connected in series with each other, and the flow channel system was prepared. These two chips comprising the flow channel were mounted on the surface plasmon resonance measurement device of the present invention.

(5) Evaluation of Binding Performance of Mouse IgG

The flow channel was filled with HBS-EP buffer (manufactured by BlAcore). The composition of the HBS-EP buffer was 0.01 mol/l (pH 7.4) HEPES (N-2-Hydroxyethylpiperazine-N′-2-ethanesulfonicAcid), 0.15 mol/l NaCl, 0.003 mol/l EDTA, and 0.005 wt. % Surfactant P20. Changes in the signal levels were assayed at intervals of 0.5 seconds by employing the signal level before liquid exchange as the baseline. The inside of the flow channel was replaced with a mouse IgG solution (a solution of 10 μg/ml mouse IgG (purchased from Cosmo Bio Co., Ltd) in HBS-EP buffer) at the rate of 20 μl/sec. The time necessary for the replacement was 5 seconds.

A compound having the following parameters was used as the aforementioned mouse IgG, and its binding curve was measured.
Ka=31500, Kd=0.0008, D=4.884E-08, C=6.67-E08, Rmax=620

Ka and D were varied in four steps to −20%, −10%, 0, and +10%, thereby obtaining a total of 16 tables.

TABLE 1
List of parameters used for the creation of tables

| No. | File name | Ka | Kd | D | C | Comments |
|---|---|---|---|---|---|---|
| 25 | Test07_00025.txt | 245000 | 0.0008 | 4.07E−08 | 6.67E−08 | Ka: −20%, D: −20% |
| 26 | Test07_00026.txt | 245000 | 0.0008 | 4.477E−08 | 6.67E−08 | Ka: −20%, D: −10% |
| 27 | Test07_00027.txt | 245000 | 0.0008 | 4.884E−08 | 6.67E−08 | Ka: −20% |
| 28 | Test07_00028.txt | 245000 | 0.0008 | 5.291E−08 | 6.67E−08 | Ka: −20%, D: +10% |
| 74 | Test07_00074.txt | 280000 | 0.0008 | 4.07E−08 | 6.67E−08 | Ka: −10%, D: −20% |
| 75 | Test07_00075.txt | 280000 | 0.0008 | 4.48E−08 | 6.67E−08 | Ka: −10%, D: −10% |
| 76 | Test07_00076.txt | 280000 | 0.0008 | 4.88E−08 | 6.67E−08 | Ka: −10% |
| 77 | Test07_00077.txt | 280000 | 0.0008 | 5.29E−08 | 6.67E−08 | Ka: −10%, D: +10% |
| 123 | Test07_00123.txt | 315000 | 0.0008 | 4.07E−08 | 6.67E−08 | D: −20% |
| 124 | Test07_00124.txt | 315000 | 0.0008 | 4.477E−08 | 6.67E−08 | D: −10% |
| 125 | Test07_00125.txt | 315000 | 0.0008 | 4.884E−08 | 6.67E−08 | |
| 126 | Test07_00126.txt | 315000 | 0.0008 | 5.291E−08 | 6.67E−08 | D: +10% |
| 172 | Test07_00172.txt | 350000 | 0.0008 | 4.07E−08 | 6.67E−08 | Ka: +10%, D: −20% |
| 173 | Test07_00173.txt | 350000 | 0.0008 | 4.477E−08 | 6.67E−08 | Ka: +10%, D: −10% |
| 174 | Test07_00174.txt | 350000 | 0.0008 | 4.884E−08 | 6.67E−08 | Ka: +10% |
| 175 | Test07_00175.txt | 350000 | 0.0008 | 5.291E−08 | 6.67E−08 | Ka: +10%, D: +10% |

Averages of the squares of the error between the table data and the measured data were calculated, and the parameters with the minimum error were selected (No. 125 in table 2). The parameters corresponded to the parameters of the compound.

TABLE 2
Averages of the squares of the error, and Rmax obtained by calculation

| | Averages of the squares | | | | | | | |
|---|---|---|---|---|---|---|---|---|
| No | of the error | Rmax | File name | Ka | Kd | D | C | Comments |
| 25 | 98.58526048 | 698.6030343 | test07_00025.txt | 245000 | 0.0008 | 4.07E−08 | 6.67E−08 | Ka: −20%, D: −20% |
| 26 | 107.6472226 | 684.5636714 | test07_00026.txt | 245000 | 0.0008 | 4.477E−08 | 6.67E−08 | Ka: −20%, D: −10% |
| 27 | 109.0962589 | 669.5979179 | test07_00027.txt | 245000 | 0.0008 | 4.884E−08 | 6.67E−08 | Ka: −20% |
| 28 | 97.61845576 | 651.4299185 | test07_00028.txt | 245000 | 0.0008 | 5.291E−08 | 6.67E−08 | Ka: −20%, D: +10% |
| 74 | 35.8618269 | 670.6198532 | test07_00074.txt | 280000 | 0.0008 | 4.07E−08 | 6.67E−08 | Ka: −10%, D: −20% |
| 75 | 38.01366394 | 655.2224359 | test07_00075.txt | 280000 | 0.0008 | 4.48E−08 | 6.67E−08 | Ka: −10%, D: −10% |
| 76 | 59.71351293 | 645.5910425 | test07_00076.txt | 280000 | 0.0008 | 4.88E−08 | 6.67E−08 | Ka: −10% |
| 77 | 43.81613033 | 633.039435 | test07_00077.txt | 280000 | 0.0008 | 5.29E−08 | 6.67E−08 | Ka: −10%, D: +10% |
| 123 | 8.855996022 | 651.6429406 | test07_00123.txt | 315000 | 0.0008 | 4.07E−08 | 6.67E−08 | D: −20% |
| 124 | 8.194388966 | 635.1367261 | test07_00124.txt | 315000 | 0.0008 | 4.477E−08 | 6.67E−08 | D: −10% |
| 125 | 7.595450658 | 621.033631 | test07_00125.txt | 315000 | 0.0008 | 4.884E−08 | 6.67E−08 | |
| 126 | 9.915381809 | 612.8560516 | test07_00126.txt | 315000 | 0.0008 | 5.291E−08 | 6.67E−08 | D: +10% |
| 172 | 2.218096583 | 634.0367941 | test07_00172.txt | 350000 | 0.0008 | 4.07E−08 | 6.67E−08 | Ka: +10%, D: −20% |
| 173 | 1.119935922 | 620.2341745 | test07_00173.txt | 350000 | 0.0008 | 4.477E−08 | 6.67E−08 | Ka: +10%, D: −10% |
| 174 | 1.106765011 | 605.9518815 | test07_00174.txt | 350000 | 0.0008 | 4.884E−08 | 6.67E−08 | Ka: +1.0% |
| 175 | 1.564479375 | 597.3596388 | test07_00175.txt | 350000 | 0.0008 | 5.291E−08 | 6.67E−08 | Ka: +10%, D: +10% |

FIG. 3 shows the individual items of table data and the binding curve. Although not clearly seen from the overall chart, it can be understood from an enlarged view in FIG. 4 which table data is closest to the measurement data

Effect of the Invention

In accordance with the method of the invention, reaction curve simulation data is retained in tables, whereby solutions of Ka, Kd, D or Rmax can be calculated more speedily than in a case where they are calculated each time they need to be calculated. Further, in accordance with the method of the invention, by examining the level of correspondence between simulation data and actually measured reaction curve, improved accuracy can be obtained as compared with the method of calculating characteristics values from a reaction curve.

1. A method for measuring adsorption rate coefficient (Ka) and diffusion coefficient (D) in a reaction between an analyte molecule immobilized on a metal surface and a molecule that interacts with the analyte molecule, by measuring an angular change in the total reflection attenuation angle (θSP) using an analysis device utilizing total reflection attenuation, which comprises
(1) providing multiple simulation curves of a binding dissociation reaction for sets of variables in which adsorption rate coefficient (Ka) and diffusion coefficient (D) are each varied within a predetermined width,
(2) preparing a measurement curve of the binding dissociation reaction based on an angular change in a measured total reflection attenuation angle (θSP),
(3) examining the level of correspondence between the measurement curve prepared in above (2) and the multiple simulation curves of above (1), and
(4) applying the adsorption rate coefficient (Ka) and diffusion coefficient (D) that were used for the preparation of the simulation curve with the highest level of correspondence to the adsorption rate coefficient (Ka) and the diffusion coefficient (D) in the reaction between the analyte molecule immobilized on the metal surface and the molecule that interacts with the analyte molecule.

2. The method according to claim 1, wherein in the step (1), multiple simulation curves of a binding dissociation reaction are provided for sets of variables in which adsorption rate coefficient (Ka), dissociation rate coefficient (Kd), diffusion coefficient (D), theoretical maximum binding amount (Rmax), and C (analyte concentration) are each varied within a predetermined width.

3. The method according to claim 1, wherein the level of correspondence between the measurement curve and the simulation curve is examined by using a square sum of the error as an index.

4. The method according to claim 1, wherein, using an analysis device utilizing total reflection attenuation and comprising a flow channel system having a cell formed on a metal film and a light-detecting means for detecting a total reflection attenuation angle (θSP) by measuring the intensity of a light beam totally reflected on the metal film, an angular change in the total reflection attenuation angle (θSP) is measured in a state where the liquid flow is stopped, after liquids in the flow channel are exchanged.

5. The method according to claim 1, wherein there is used a analysis device utilizing total reflection attenuation, which comprises a dielectric block, a metal film formed on one side of the dielectric block, a light source for generating a light beam, an optical system for allowing the above light beam to enter the above dielectric block so that total reflection conditions can be obtained at the interface between the dielectric block and the metal film and so that various incidence angles can be included, a flow channel system comprising a cell formed on the above metal film, and a light-detecting means for detecting a total reflection attenuation angle (θSP) by measuring the intensity of a light beam totally reflected at the above interface.

6. The method according to claim 1, wherein the liquid contained in the above flow channel system is exchanged from a reference liquid containing no test substance to be measured to a sample liquid containing a test substance to be measured, and thereafter an angular change in the total reflection attenuation angle (θSP) is measured in a state where the sample liquid flow is stopped.

7. The method according to claim 1, wherein, as the range in which the level of correspondence between the measurement curve and the simulation curve is examined, a part of a binding signal curve, a part of a dissociation signal curve, or both thereof is used.

8. The method according to claim 1, wherein a signal change in surface plasmon resonance is measured by using a surface plasmon resonance measurement device.
